SEQUENCE LISTING (10 sequences)

SEQ ID NO: 1 - DNA, 954 bp, Homo sapiens
aataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaac

SEQ ID NO: 2 - DNA, 21 bp, Artificial sequence
  Synthetic construct
gagtgcttagttgaacagggc

SEQ ID NO: 3 - DNA, 20 bp, Artificial sequence
  Synthetic construct
gggtttggggctaggtttag

SEQ ID NO: 4 - DNA, 25 bp, Artificial sequence
  Synthetic construct
  misc_feature (25)..(25): n is T modified with disulfide amide
cgacttgtctcctcttttttttttn

SEQ ID NO: 5 - DNA, 25 bp, Artificial sequence
  Synthetic construct
  misc_feature (25)..(25): n is T modified with disulfide amide
cgacttgcctcctcttttttttttn

SEQ ID NO: 6 - DNA, 25 bp, Artificial sequence
  Synthetic construct
  misc_feature (25)..(25): n is T modified with disulfide amide
ttgaggagggtgacgtttttttttn

SEQ ID NO: 7 - DNA, 25 bp, Artificial sequence
  Synthetic construct
  misc_feature (25)..(25): n is T modified with disulfide amide
ttgaggagagtgacgtttttttttn

SEQ ID NO: 8 - DNA, 100 bp, Artificial sequence
  Synthetic construct
gtacacaccgcccgtcaccctcctcaagtatacttcaaaggacatttaactaaaacccctacgcatttatatagaggagacaagtcgtaacatggtaagt

SEQ ID NO: 9 - DNA, 100 bp, Artificial sequence
  Synthetic construct
gtacacaccgcccgtcaccctcctcaagtatacttcaaaggacatttaactaaaacccctacgcatttatatagaggaggcaagtcgtaacatggtaagt

SEQ ID NO: 10 - DNA, 100 bp, Artificial sequence
  Synthetic construct
gtacacaccgcccgtcactctcctcaagtatacttcaaaggacatttaactaaaacccctacgcatttatatagaggagacaagtcgtaacatggtaagt

(19) United States
(12) Patent Application Publication
(10) Pub. No.: US 2020/0165672 A1
(43) Pub. Date: May 28, 2020

(54) DETERMINATION OF MTRNR1 GENE MUTATION

(71) Applicant: Agency for Science, Technology and Research, Singapore (SG)

(72) Inventors: Yanbing Zu, Singapore (SG); Jackie Y. Ying, Singapore (SG)

(21) Appl. No.: 15/999,372

(22) PCT Filed: Feb. 9, 2017
(86) PCT No.: PCT/SG2017/050057

§ 371 Date: Aug. 17, 2018

(30) Foreign Application Priority Data
Feb. 17, 2016 (SG) 10201601165V

(51) Int. Cl. C12Q 1/6883

(52) U.S. Cl. CPC C12Q 1/6883; C12Q 2600/118; C12Q 2600/156

(57) ABSTRACT

The invention relates to methods and kits for determining the presence of a mutation selected from the group consisting of 1555A>G and 1494C>T in human Mitochondrially Encoded 12S RNA (MTRNR1) gene in a sample from a subject, comprising, inter alia, amplifying at least part of the MTRNR1 gene and detecting the amplified DNA with a plasmonic gold nanoparticle covalently coupled to a morpholino oligonucleotide probe. The claimed method may be used for determining the risk of a subject to Aminoglycoside-induced hearing loss.

This application makes reference to and claims the benefit of priority of the Singapore Patent Application No. 10201601165V filed on Feb. 17, 2016, the content of which is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates generally to methods and kits for detection of the presence of a mutation in human Mitochondrially Encoded 12S RNA (MTRNR1) gene (SEQ ID NO:1).

### BACKGROUND OF THE INVENTION

Genetic mutations are the main causes of early childhood hearing loss. Two pathogenic variants in the human mitochondrial gene MTRNR1 (12S rRNA), namely 1555A>G and 1494C>T, are known to be closely related to deafness induced by exposure to aminoglycosides. Such mutations are also maternally transmitted from a woman to all of her off-springs.

Aminoglycosides, including streptomycin, gentamicin, kanamycin, tobramycin and neomycin, are antibiotics commonly used to treat bacterial infections. However, these antibiotics are also well known to be ototoxic. Individuals bearing mutations of mtDNA 1555A>G or 1494C>T may suffer from rapidly progressive, profound and permanent hearing loss if they are given aminoglycoside antibiotics, even when drug levels are within normal limits.

Therefore, genetic screening of the pathogenic mtDNA variants prior to treatment with aminoglycosides may provide information about the risk of aminoglycoside-induced hearing loss. By choosing alternative therapy, hearing loss may be prevented in predisposed individuals.

Various technologies have been existing in the prior art for the determination of gene mutation. However, there still remains a considerable need for new technologies to overcome the drawbacks of existing technologies.

### SUMMARY OF THE INVENTION

The present invention satisfies the aforementioned need in the art by providing a new method and kit for determining the presence of a mutation in human MTRNR1 gene (SEQ ID NO:1).

In one aspect, the present invention provides a method of determining the presence of a mutation in human MTRNR1 gene (SEQ ID NO:1) in a sample, wherein the mutation is selected from the group consisting of 1555A>G and 1494C>T, and wherein the method comprises the steps of:
(a) amplifying at least part of the MTRNR1 gene, said part comprising the locus the mutation status of which is to be analyzed, in a polymerase chain reaction (PCR), using a pair of primers under conditions allowing such amplification to generate PCR amplification products (amplicons);
(b) contacting the amplicons in different test tubes with a plasmonic nanoprobe specific for the wild-type or mutant amplicons, respectively, said plasmonic nanoprobe comprising a plasmonic nanoparticle and a non-ionic oligonucleotide analog probe covalently coupled thereto, the oligonucleotide analog probe comprising a base sequence that is complementary to the wild-type or mutant amplicons, under conditions that allow the oligonucleotide analog probe and the amplicons to hybridize to each other, wherein the probe generates a detectable signal if hybridized to the amplicons that is distinguishable from the signal of the unhybridized probe;
(c) determining the presence of the mutation based on the determination of the melting temperature Tₘ of the hybrid of the nanoprobe and the amplicons.

In various embodiments, the PCR used in step a) of the method is asymmetric PCR (aPCR).

In various embodiments, the plasmonic nanoparticle comprised in the plasmonic nanoprobe as used in step b) of the method is a plasmonic gold nanoparticle.

In various embodiments, the non-ionic oligonucleotide analog probe comprised in the plasmonic nanoprobe as used in step b) of the method is a morpholino oligonucleotide (MOR) probe.

In various embodiments, the detectable signal generated in step b) of the method is the color of the assay solution that is indicative of whether the probe is hybridized to the amplicons or not.

In various embodiments, the melting temperature determined in step c) is indicated by a color change caused by nanoprobe dissociation and subsequent aggregation.

In various embodiments, the method further comprises isolating genomic DNA from the sample prior to step (a) of the method.

In various embodiments, the method comprises using a nucleic acid molecule comprising at least part of the MTRNR1 gene comprising the 1555A>G and 1494C>T mutations as a positive control, and/or using a nucleic acid molecule comprising at least part of the MTRNR1 gene without said mutations as a negative control.

In various embodiments, the PCR primers for use in the method have the nucleic acid sequences 5′-GAGTGCTTAGTTGAACAGGGC-3′ (SEQ ID NO:2) and 5′-GGGTTTGGGGCTAGGTTTAG-3′ (SEQ ID NO:3), and the oligonucleotide analog probes used are morpholino oligonucleotides having the nucleic acid sequence 5′-CGACTTGTCTCCTCTTTTTTTTTTT-3′ (SEQ ID NO:4) (specific for 1555A>G WT), 5′-CGACTTGCCTCCTCTTTTTTTTTTT-3′ (SEQ ID NO:5) (specific for 1555A>G MUT), 5′-TTGAGGAGGGTGACGTTTTTTTTTT-3′ (SEQ ID NO:6) (specific for 1494C>T WT) or 5′-TTGAGGAGAGTGACGTTTTTTTTTT-3′ (SEQ ID NO:7) (specific for 1494C>T MUT).

In various embodiments, the morpholino oligonucleotides are modified with disulfide amide at the 3′ terminal.

In various embodiments, the method is for use in determining the predisposition of a subject to a disease or disorder associated with the mutations of the MTRNR1 gene such as determining the risk of the subject to Aminoglycoside-induced hearing loss.

In another aspect, the invention provides a kit for determining the presence of a mutation in the MTRNR1 gene (SEQ ID NO:1) in a sample, wherein the mutation is selected from the group consisting of 1555A>G and 1494C>T, and wherein the kit comprises a pair of PCR primers and a pair of plasmonic nanoprobes for use in the method disclosed herein.

In various embodiments, the kit is designed to determine both of the 1555A>G and 1494C>T mutations and thus comprises four plasmonic nanoprobes.

In various embodiments, the kit comprises a pair of PCR primers having the nucleic acid sequences 5′-GAGTGCTTAGTTGAACAGGGC-3′ (SEQ ID NO:2) and 5′-GGGTTTGGGGCTAGGTTTAG-3′ (SEQ ID NO:3), and four plasmonic gold nanoparticle each respectively functionalized with a morpholino oligonucleotide having the nucleic acid sequence 5′-CGACTTGTCTCCTCTTTTTTTTTTT-3′ (SEQ ID NO:4) (specific for 1555A>G WT), 5′-CGACTTGCCTCCTCTTTTTTTTTTT-3′ (SEQ ID NO:5) (specific for 1555A>G MUT), 5′-TTGAGGAGGGTGACGTTTTTTTTTT-3′ (SEQ ID NO:6) (specific for 1494C>T WT) or 5′-TTGAGGAGAGTGACGTTTTTTTTTT-3′ (SEQ ID NO:7) (specific for 1494C>T MUT), said morpholino oligonucleotides being modified with disulfide amide at the 3′ terminal.

In various embodiments, the kit is for use in determining the predisposition of a subject to a disease or disorder associated with the mutations of the MTRNR1 gene such as determining the risk of the subject to Aminoglycoside-induced hearing loss.

### BRIEF DESCRIPTION OF THE DRAWINGS

The invention will be better understood with reference to the detailed description when considered in conjunction with the non-limiting examples and the accompanying drawings.

FIG. 1 shows the melting temperature as a function of target concentration for the (a) WT and (b) MUT probes targeting mtDNA 1555A>G. Samples are synthetic single-stranded WT DNA (SEQ ID NO:8) and MUT DNA (SEQ ID NO:9). Error of Tₘ measurement=±1° C.

FIG. 2 shows the melting temperature as a function of target concentration for the (a) WT and (b) MUT probes targeting mtDNA 1494C>T. Samples are synthetic single-stranded WT DNA (SEQ ID NO:8) and MUT DNA (SEQ ID NO:10). Error of Tₘ measurement=±1° C.

FIG. 3 shows scatter plots of Tₘ^{WT} and Tₘ^{MUT} for genotyping of (a) mtDNA 1555A>G and (b) mtDNA 1494C>T. Error of Tₘ measurement=±1° C.

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

The object of the present invention is to provide a method of determining the presence of a mutation in human MTRNR1 gene (SEQ ID NO:1).

To this end, the inventors of the present invention have provided such a method employing polymerase chain reaction (PCR) and plasmonic nanoprobe-based detection.

In one aspect, disclosed herein is a method of determining the presence of a mutation in human MTRNR1 gene (SEQ ID NO:1) in a sample, wherein the mutation is selected from the group consisting of 1555A>G and 1494C>T, and wherein the method comprises the steps of:
(a) amplifying at least part of the MTRNR1 gene, said part comprising the locus the mutation status of which is to be analyzed, in a polymerase chain reaction (PCR), preferably asymmetric PCR (aPCR), using a pair of primers under conditions allowing such amplification to generate PCR amplification products (amplicons);
(b) contacting the amplicons in different test tubes with a plasmonic nanoprobe specific for the wild-type (WT) or mutant (MUT) amplicons, respectively, said plasmonic nanoprobe comprising a plasmonic nanoparticle, preferably a plasmonic gold nanoparticle, and a non-ionic oligonucleotide analog probe, preferably morpholino oligonucleotide (MOR) probe, covalently coupled thereto, the oligonucleotide analog probe comprising a base sequence that is complementary to the wild-type or mutant amplicons, under conditions that allow the oligonucleotide analog probe and the amplicons to hybridize to each other, wherein the probe generates a detectable signal if hybridized to the amplicons that is distinguishable from the signal of the unhybridized probe, wherein said detectable signal is preferably the color of the assay solution that is indicative of whether the probe is hybridized to the amplicons or not;
(c) determining the presence of the mutation based on the determination of the melting temperature Tₘ of the hybrid of the nanoprobe and the amplicons, wherein the melting temperature is preferably indicated by a color change caused by nanoprobe dissociation and subsequent aggregation.

The complete sequence of the human MTRNR1 gene, as set forth in SEQ ID NO:1, spans from position 648 to position 1601 of the human mitochondrial genome (GenBank accession number: NC_012920.1). The terms “mutation” or “gene mutation” as used herein refers to an alteration in the base sequence of a DNA strand compared to the wild-type reference strand. More specifically, 1555A>G refers to the mutation of A→G at position 1555 of the human mitochondrial genome and 1494C>T refers to the mutation of C→T at position 1494 of the human mitochondrial genome. It is to be understood that in the context of the present invention, said terms include the term “polymorphism” or any other similar or equivalent term of art.

The term “sample” as used herein refers to anything capable of being analyzed by the methods described herein. Samples can include, for example, purified DNA, cells, blood, semen, saliva, urine, feces, rectal swabs, and the like.

In certain embodiments, the method disclosed herein may further comprise isolating genomic DNA from the sample prior to step (a).

The method described herein employs PCR for the specific amplification of at least part of the MTRNR1 gene comprising the locus the mutation status of which is to be analyzed, and plasmonic nanoprobe-based detection of the resultant amplicons, and thus can be used to determine the mutation status of the MTRNR1 gene.

Any PCR that may produce single-stranded amplicons for hybridization to the oligonucleotide analog probe of the plasmonic nanoprobes may be used in the present method. Such types of PCR technology include, but are not limited to allele-specific PCR, assembly PCR, asymmetric PCR, dial-out PCR, digital PCR, helicase-dependent amplification, hot start PCR, intersequence-specific PCR (ISSR), inverse PCR, ligation-mediated PCR, methylation-specific PCR (MSP), miniprimer PCR, multiplex ligation-dependent probe amplification (MLPA), multiplex-PCR, nanoparticle-assisted PCR (nanoPCR), nested PCR, overlap-extension PCR or splicing by overlap extension (SOEing), PAN-AC, reverse transcription PCR (RT-PCR), solid phase PCR, thermal asymmetric interlaced PCR (TAIL-PCR), touchdown PCR (step-down PCR), universal fast walking or transcription-mediated amplification (TMA). Such techniques are well-known in the art (McPherson, M J and Moller, S G (2000) PCR (Basics), Springer-Verlag Telos; first edition).

In preferred embodiments, asymmetric PCR (aPCR) is used. aPCR is a PCR wherein the amounts of the two primers are unequal. The primer present at a higher amount is referred to as the excess primer, and the strand resulting from the extension of the excess primer is accumulated in excess and is hybridized subsequently to the oligonucleotide analog probe of the plasmonic nanoprobe of the invention.

The PCR amplicons are further contacted with a plasmonic nanoprobe specific for the wild-type or mutant amplicons, respectively, said plasmonic nanoprobe comprising a plasmonic nanoparticle and a non-ionic oligonucleotide analog probe covalently coupled thereto, the oligonucleotide analog probe comprising a base sequence that is complementary to the wild-type or mutant amplicons, under conditions that allow the oligonucleotide analog probe and the amplicons to hybridize to each other.

Without wishing to be bound to any particular theory, the probe in accordance with the present invention generates a detectable signal if hybridized to the amplicons that is distinguishable from the signal of the unhybridized probe. Said signal may be any signal that is detectable by any means.

In preferred embodiments, these probes indicate the presence or absence of the target by showing a color (e.g. red) in their hybridized state and another color (e.g. light grey) in their unhybridized, aggregated state. The aggregation of the unhybridized nanoprobes may generally be achieved by control of the ionic strength of the assay solution, for example by control of salt concentrations. This particular behavior of the nanoprobes, i.e. remaining in non-aggregated form as long as they are hybridized to their target and aggregated if not hybridized to their target, can be attributed to the non-ionic character of the nanoprobes.

The hybridization of the plasmonic nanoprobe and the amplicons in step b) of the method may be carried out at a temperature below the melting temperature of the duplex of the nanoprobe and the amplicons having a perfect complementarity, and above that of the duplex of the nanoprobe and the amplicons having an imperfect complementarity, to allow maximum distinction between these two groups. In these embodiments, step c) may also be carried out at the above-described temperature by simply determining the color of the assay solution that is indicative of whether the hybrid has been formed or not. In these embodiments, the melting temperature of the hybrid is thus only determined insofar as it is determined whether the formed hybrid has a melting temperature above the assay temperature, indicating the presence of the amplicons perfectly complementary to the plasmonic nanoprobe, or a melting temperature below the assay temperature, indicating the absence of the amplicons perfectly complementary to the plasmonic nanoprobe.

The term “nanoparticle” as used herein refers to any particle having a size from about 1 to about 250 nm and has the capacity to be covalently coupled to at least one oligonucleotide analog as described herein. In certain embodiments, the nanoparticle is a metal nanoparticle. In other embodiments, the nanoparticle is a colloidal metal.

In some embodiments, the metal is a noble metal. Non-limiting examples of a noble metal that can be used can include silver, gold, platinum, palladium, ruthenium, osmium, iridium or mixtures thereof, not to mention a few. Other metals that can also be used in the formation of the nanoparticle can include but are not limited to aluminium, copper, cobalt, indium, nickel, or any other metal amenable to nanoparticle formation). The nanoparticle as described herein can also comprise a semiconductor (including for example and without limitation, CdSe, CdS, and CdS or CdSe coated with ZnS) or magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs.

The size of the nanoparticle used in the conjugate of the present invention can vary in any size when desired, as long as the nanoparticle is capable of providing optical properties; for example, generate optical signals sensitive to hybridization reactions. The diameter of the nanoparticle as described herein can range in the size from about 1 nm to about 250 nm; about 1 nm to about 200 nm; about 1 nm to about 160 nm; about 1 nm to about 140 nm; about 1 nm to about 120 nm; about 1 nm to about 80 nm; about 1 nm to about 60 nm; about 1 nm to about 50 nm; about 5 nm to about 250 nm; about 8 nm to about 250 nm; about 10 nm to about 250 nm; about 20 nm to about 250 nm; about 30 nm to about 250 nm; about 40 nm to about 250 nm; about 85 nm to about 250 nm; about 100 nm to about 250 nm; or about 150 nm to about 250 nm. In some embodiments, the diameter of the diameter of the nanoparticle is in the range of about 1 nm to about 100 nm.

In certain embodiments, the nanoparticle comprises a surfactant. As used herein, “surfactant” refers to a surface active agent which has both hydrophilic and hydrophobic parts in the molecule. The surfactant can for example be used to stabilize the nanoparticles. The surfactant can also be used to prevent non-specific adsorption of the oligonucleotide analog on the surface of the nanoparticles. In some embodiments, the surfactant is a non-ionic surfactant. Other types of surfactants that can be used can include but are not limited to cationic, anionic, or zwitterionic surfactants. A particular surfactant may be used alone or in combination with other surfactants. One class of surfactants comprises a hydrophilic head group and a hydrophobic tail. Hydrophilic head groups associated with anionic surfactants include carboxylate, sulfonate, sulfate, phosphate, and phosphonate. Hydrophilic head groups associated with cationic surfactants include quaternary amine, sulfonium, and phosphonium. Quaternary amines include quaternary ammonium, pyridinium, bipyridinium, and imidazolium. Hydrophilic head groups associated with non-ionic surfactants include alcohol and amide. Hydrophilic head groups associated with zwitterionic surfactants include betaine. The hydrophobic tail typically comprises a hydrocarbon chain. The hydrocarbon chain typically comprises between about six and about 24 carbon atoms, more typically between about eight to about 16 carbon atoms.

The plasmonic nanoparticle for use in the present method is functionalized with a non-ionic oligonucleotide analog probe that preferably recognizes the amplicons to be analyzed. For the practice of the present invention, a total of four plasmonic nanoparticle may be simultaneously used to determine the presence of the two mutations.

In some embodiments, the non-ionic oligonucleotide analog probe used in the presently disclosed method is a morpholino oligonucleotide probe or a derivative thereof The term “oligonucleotide analog” refers to an oligonucleotide having (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. The analog supports bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). The analogs can for example, include those having a substantially uncharged, phosphorus containing backbone.

A substantially uncharged, phosphorus containing backbone in an oligonucleotide analog can for example be one in which a majority of the subunit linkages, e.g., between 60-100%, are uncharged at physiological pH, and contain a single phosphorous atom. The oligonucleotide analog can comprise a nucleotide sequence complementary to a target amplicon as defined below. In preferred embodiments, the oligonucleotide analogs of the present invention are phosphorodiamidate morpholino oligos, wherein the sugar and phosphate backbone is replaced by morpholine groups linked by phosphoramidates and the nucleobases, such as cytosine, guanine, adenine, thymine and uracil, are coupled to the morpholine ring or derivatives thereof.

As used herein, the term “complementary” or “complementarity” relates to the relationship of nucleotides/bases on two different strands of DNA or RNA, or the relationship of nucleotides/bases of the nucleotide sequence of the oligonucleotide analog probe and a DNA/RNA strand, where the bases are paired (for example by Watson-Crick base pairing: guanine with cytosine, adenine with thymine (DNA) or uracil (RNA)). Therefore, the oligonucleotide analog probe as described herein can comprise a nucleotide sequence that can form hydrogen bond(s) with another nucleotide sequence, for example a DNA or RNA sequence, by either conventional Watson-Crick base pairing or other non-traditional types of pairing such as Hoogsteen or reversed Hoogsteen hydrogen bonding between complementary nucleosides or nucleotides. In this context, the term “hybridize” or “hybridization” refers to an interaction between two different strands of DNA or RNA or between nucleotides/bases of the nucleotide sequence of the oligonucleotide analog probe and a DNA/RNA sequence by hydrogen bonds in accordance with the rules of Watson-Crick DNA complementarity, Hoogsteen binding, or other sequence-specific binding known in the art. In this context, it is understood in the art that a nucleotide sequence of an oligonucleotide analog described herein need not be 100% complementary to a target nucleic acid sequence to be specifically or selectively hybridizable.

Complementarity is indicated by a percentage of contiguous residues in a nucleic acid molecule that can form hydrogen bonds with a second nucleic acid molecule. For example, if a first nucleic acid molecule has 10 nucleotides and a second nucleic acid molecule has 10 nucleotides, then base pairing of 5, 6, 7, 8, 9, or 10 nucleotides between the first and second nucleic acid molecules represents 50%, 60%, 70%, 80%, 90%, or 100% complementarity, respectively, not to mention a few.

Therefore, in some embodiments, the oligonucleotide analog used herein can be 100% complementary to a target amplicon (i.e., a perfect match). In other embodiments, the oligonucleotide analog probe can be at least about 95% complementary, at least about 85% complementary, at least about 70% complementary, at least about 65% complementary, at least about 55% complementary, at least about 45% complementary, or at least about 30% complementary to the target amplicon, provided that it can specifically recognizes the intended target amplicon over the unintended amplicon.

The length of the oligonucleotide analog probe described herein can comprise about 5 monomelic units to about 40 monomelic units; about 10 monomelic units to about 35 monomelic units; or about 15 monomelic units to about 35 monomelic units. The term “monomeric unit” of an oligonucleotide analog probe as used herein refers to one nucleotide unit of the oligonucleotide analog.

In certain embodiments, the oligonucleotide analog probe is covalently coupled to the nanoparticle via a functional group. The functional group is typically included in the spacer portion of the oligonucleotide analog probe for covalently binding to the nanoparticle. In some embodiments, the functional group can include a thiol (SH) group, which can for example be used to covalently attach to the surface of the nanoparticle. However, other functional groups can also be used. Oligonucleotides functionalized with thiols at their 3′-end or 5′-end can readily attach to gold nanoparticles. See for example, Mucic et al. Chem. Commun. 555-557 (1996) which describes a method of attaching 3′ thiol DNA to flat gold surfaces. The thiol moiety also can be used to attach oligonucleotides to other metal, semiconductor, and magnetic colloids and to the other types of nanoparticles described herein. Other functional groups for attaching oligonucleotides to solid surfaces include phosphorothioate groups (see, for example, U.S. Pat. No. 5,472,881 for the binding of oligonucleotide-phosphorothioates to gold surfaces), substituted alkylsiloxanes (see, for example Grabar et al., Anal. Ghent., 67, 735-743). Oligonucleotides having a 5′ thionucleoside or a 3′ thionucleoside may also be used for attaching oligonucleotides to solid surfaces. Other functional groups known to the skilled person that can be used to attach the oligonucleotide analog probe to nanoparticles can include but are not limited to disulfides such as disulfide amides; carboxylic acids; aromatic ring compounds; sulfolanes; sulfoxides; silanes, not to mention a few.

A more detailed description of the plasmonic nanoparticles and nanoprobes for the practice of the present method may be found in PCT international patent publication No. WO 2011/087456 A1, which is hereby incorporated by reference in its entirety, with the probe sequences adapted for the target of interest.

The plasmonic nanoprobes developed by the inventors of the present invention are highly specific in recognition of nucleic acid sequences. In preferred embodiments, plasmonic gold nanoparticles are functionalized with non-ionic morpholino oligonucleotides. Unlike the DNA-modified gold nanoparticles that are stably dispersed in salt solution, the non-ionic nature of the morpholino oligonucleotides makes the morpholino oligonucleotides-modified nanoparticles much less stable, and only dispersible in solutions with low ionic strength (e.g., [NaCl]<10 mmol/L). An increase of solution ionic strength would lead to solution color change from red to light grey/colorless due to nanoparticle aggregation. However, upon hybridization with negatively charged DNA molecules, the nanoprobes become much more stable due to the increase in surface charge, and the solution remains red at a high ionic strength (e.g., [NaCl]˜100 mmol/L). When temperature rises, sharp melting transition occurs at melting temperature (Tₘ), whereby DNA molecules are released from the nanoprobes, resulting in rapid color change in solution. The nanoprobes are highly specific in recognizing DNA targets, and a single-base mismatch may lead to the decrease in Tₘ by 5-12° C. This technology allows for accurate end-point detection with standard equipment and a simple workflow. The colorimetric signals can be easily visualized and recorded.

In certain embodiments, the method disclosed herein comprises using a nucleic acid molecule comprising at least part of the MTRNR1 gene comprising the 1555A>G and 1494C>T mutations as a positive control, and/or using a nucleic acid molecule comprising at least part of the MTRNR1 gene without said mutations as a negative control. The mutation status of the MTRNR1 gene in a sample can be easily determined by comparing the Tₘ data or color information thereof to the controls.

In preferred embodiments, the PCR primers for use in the method have the nucleic acid sequences 5′-GAGTGCTTAGTTGAACAGGGC-3′ (SEQ ID NO:2) and 5′-GGGTTTGGGGCTAGGTTTAG-3′ (SEQ ID NO:3), and the oligonucleotide analog probes used are morpholino oligonucleotides having the nucleic acid sequence 5′-CGACTTGTCTCCTCTTTTTTTTTTT-3′ (SEQ ID NO:4) (specific for 1555A>G WT), 5′-CGACTTGCCTCCTCTTTTTTTTTTT-3′ (SEQ ID NO:5) (specific for 1555A>G MUT), 5′-TTGAGGAGGGTGACGTTTTTTTTTT-3′ (SEQ ID NO:6) (specific for 1494C>T WT) or 5′-TTGAGGAGAGTGACGTTTTTTTTTT-3′ (SEQ ID NO:7) (specific for 1494C>T MUT). In preferred embodiments, these morpholino oligonucleotides are modified with disulfide amide at the 3′ terminal.

Further disclosed herein is a kit for determining the presence of a mutation in the MTRNR1 gene (SEQ ID NO:1) in a sample, wherein the mutation is selected from the group consisting of 1555A>G and 1494C>T, and wherein the kit comprises a pair of PCR primers and a pair of plasmonic nanoprobes as described above. In preferred embodiments, the kit is designed to determine both of said mutations and thus comprises four plasmonic nanoprobes as described above.

In certain embodiments, the kit comprises a pair of PCR primers having the nucleic acid sequences 5′-GAGTGCTTAGTTGAACAGGGC-3′ (SEQ ID NO:2) and 5′-GGGTTTGGGGCTAGGTTTAG-3′ (SEQ ID NO:3), and four plasmonic gold nanoparticle each respectively functionalized with a morpholino oligonucleotide having the nucleic acid sequence 5′-CGACTTGTCTCCTCTTTTTTTTTTT-3′ (SEQ ID NO:4) (specific for 1555A>G WT), 5′-CGACTTGCCTCCTCTTTTTTTTTTT-3′ (SEQ ID NO:5) (specific for 1555A>G MUT), 5′-TTGAGGAGGGTGACGTTTTTTTTTT-3′ (SEQ ID NO:6) (specific for 1494C>T WT) or 5′-TTGAGGAGAGTGACGTTTTTTTTTT-3′ (SEQ ID NO:7) (specific for 1494C>T MUT), said morpholino oligonucleotides being modified with disulfide amide at the 3′ terminal.

Further encompassed within the scope of the present invention are the methods and kits as described above for use in determining the predisposition of a subject to a disease or disorder associated with said mutations of the MTRNR1 gene (SEQ ID NO:1), including but not limited to determining the risk of the subject to Aminoglycoside-induced hearing loss.

Unless otherwise defined, all technical and scientific terms used herein have the same meaning as commonly understood by one of ordinary skill in the art. In case of conflict, the present document, including definitions, will control.

The present invention is further illustrated by the following examples. However, it should be understood, that the invention is not limited to the exemplified embodiments.

### EXAMPLES

### Materials and Methods

### A. Preparation of the Nanoprobes

The preparation of the nanoprobes used herein is similar to that reported previously (Zu Y, et al. Anal Chem. 2011 Jun. 1; 83(11):4090-4; Zu Y, et al. Small. 2011 Feb. 7; 7(3):306-10). Briefly, the MORs modified with disulfide amide at the 3′ terminal (Gene Tools, LLC) were treated with dithiothreitol to reduce the disulfide bond, and then purified by using an NAP-5 column (GE Healthcare). Gold NPs (40 nm-diameter, ˜0.1 nM, Ted Pella, Inc.) were mixed with ˜2 μM of thiolated MORs and 10 mM of phosphate buffer (pH 7.5), and allowed to incubate at room temperature overnight. Next, the MOR-NP conjugates were washed for at least 5 times with a phosphate buffer solution (5 mM, pH 7.5) by centrifugation to remove the unreacted MORs. The conjugates could be used immediately as nanoprobes or stored in 4° C. refrigerator until use. The nanoprobes were stable for at least 6 months when stored at 4° C. Before use, the nanoprobe solutions should be uniformly dispersed by vortexing.

B. gDNA Extraction

Human gDNA samples could be extracted from whole blood, cheek swab or saliva. The extraction could be performed with the use of the commercial kit, Gentra Puregene DNA extraction kit (Qiagen), according to the manufacturer's instruction. Quantity (ng/μl) and quality of the gDNA samples could be checked by absorbance measurements using Nanodrop 1000 (Thermo Scientific). The quality of the samples was characterized by the ratio of absorbance at 260 nm and 280 nm (A260/A280 ratio), which typically varied from 1.6 to 2.0.

C. aPCR

aPCR was used to produce single-stranded DNA targets. PCR solution with a final volume of 25 μL contained gDNA, 12.5 μL of master mix (Fermentas or Promega, 2×), 1000 nM of the forward primer, and 100 nM of the reverse primer. PCR cycling (Table 3) was performed on the PTC-200 DNA Engine (Bio-Rad). The success of the PCR in producing specifically sized amplicons was verified by running a 5-μL aliquot of the PCR products on a 1.5% agarose gel stained with SafeView™ dye.

### D. Tₘ Measurements

The synthetic targets or PCR amplicons were simply mixed with the specific WT and MUT nanoprobes. Next, the Tₘ values of the target-probe hybrids were measured with the thermal cycler. The temperature was increased from 32° C. at an interval of 1.0° C. At each temperature, the solution was allowed to incubate for 1 min prior to the color visualization or recording with a camera. When a clear color change from red to light grey was observed, the temperature was recorded as Tₘ.

### E. Genotype Assignment

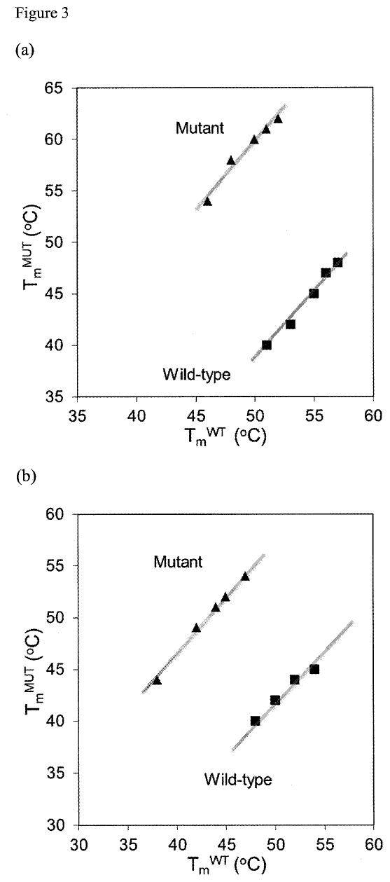

The Tₘ^{WT}-Tₘ^{MUT} scatter plots obtained with the synthetic DNA targets (FIG. 3) were used as the standard genotyping diagrams. The experimental data point (Tₘ^{WT}, Tₘ^{MUT}) of samples could be plotted in the diagram, and the genotype could be easily determined by the region where the data point resided.

### Example: Detection of Human MTRNR1 Gene Mutations

Disclosed herein are a method and an assay kit for determining two mtDNA mutations (1555A>G and 1494C>T) of the MTRNR1 gene related to aminoglycoside-induced hearing loss. The genetic test was conducted using a dual-nanoprobe-based method recently developed in the laboratory of the inventors of the present invention (Zu, et al., Small, 7 (2011) 306-310; Zu, et al., Nano Today, 9 (2014) 166-171). The nanoprobes used were highly specific, and their plasmonic properties allowed for colorimetric detection.

For each mutation assay, two sets of nanoprobes, i.e., wild-type (WT) and mutant (MUT) probes, were used. The nanoprobes were prepared by functionalizing gold nanoparticles with morpholino oligonucleotides (MORs). The oligo sequences are shown in Table 1. The oligo sequence of the WT nanoprobe was perfectly matched with the WT gene segment, while the oligo sequence of the MUT nanoprobe was perfectly matched with the mutant allele. The nanoprobes were stably dispersed in 5 mM of phosphate buffer as a red solution (pH ˜8) for at least 6 months. However, the addition of 100 mM of NaCl would lead to irreversible aggregation of the nanoparticles, and the solution color would turn colorless within 1 min.

TABLE 1
Sequences of the MORs used in this work.
The single-base differences for each pair of
the probes are underlined.

| SEQ ID | | |
|---|---|---|
| NO: | MOR probe | Sequence (5′ to 3′) |
| 4 | mtDNA | CGACTTGTCTCCTCTTTTTTTTTTT- |
| | 1555A > G WT | disulfide amide |
| 5 | mtDNA | CGACTTGCCTCCTCTTTTTTTTTTT- |
| | 1555A > G MUT | disulfide amide |
| 6 | mtDNA | TTGAGGAGGGTGACGTTTTTTTTTT- |
| | 1494C > T WT | disulfide amide |
| 7 | mtDNA | TTGAGGAGAGTGACGTTTTTTTTTT- |
| | 1494C > T MUT | disulfide amide |

The presence of the target DNA in the solution could increase the stability of the nanoprobes due to the increase in surface negative charge of the nanoparticles upon DNA attachment. If the surface density of attached DNA was high enough, the nanoparticles would be stably dispersed even in the presence of 100 mM of NaCl. To reveal the thermodynamic property of the DNA-nanoprobe hybrids, melting temperature (Tₘ) was measured. At Tₘ, the dissociation of the DNA sequence from the nanoparticle surface would occur, destabilizing the nanoparticles and resulting in solution color change from red to light grey. The Tₘ data were then used to determine the genotype of the samples.

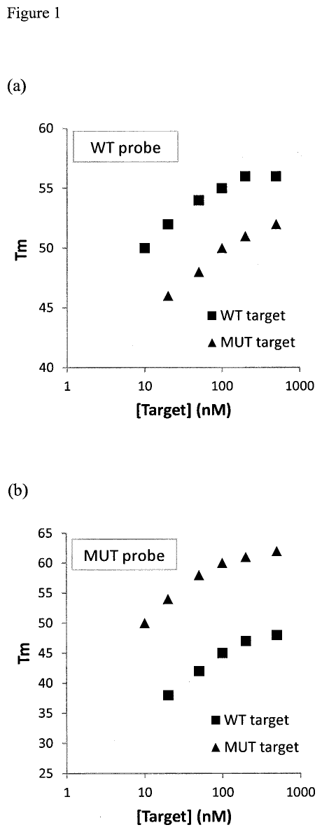

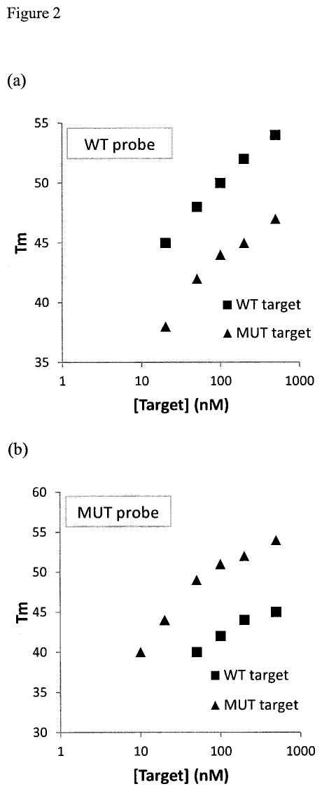

To characterize the nanoprobes, Tₘ data were measured in the presence of 100 mM of NaCl (final concentration) and synthetic DNA samples over a broad concentration range of 5 nM to 500 nM (FIGS. 1 and 2, and Table 2). The Tₘ difference induced by a single-base mismatch between the target and the probe was ˜6-15° C., allowing for clear differentiation.

TABLE 2
Sequences of the synthetic DNA used herein.
The mutation positions are underlined.

| SEQ ID | Synthetic DNA | |
|---|---|---|
| NO: | samples | Sequence (5′ to 3′) |
| 8 | mtDNA target | GTACACACCGCCCGTCACCCTCCTCAA |
| | WT | GTATACTTCAAAGGACATTTAACTAAA |
| | | ACCCCTACGCATTTATATAGAGGAGAC |
| | | AAGTCGTAACATGGTAAGT |
| 9 | mtDNA target | GTACACACCGCCCGTCACCCTCCTCAA |
| | 1555A > G MUT | GTATACTTCAAAGGACATTTAACTAAA |
| | | ACCCCTACGCATTTATATAGAGGAGGC |
| | | AAGTCGTAACATGGTAAGT |
| 10 | mtDNA target | GTACACACCGCCCGTCACTCTCCTCAA |
| | 1494C > T MUT | GTATACTTCAAAGGACATTTAACTAAA |
| | | ACCCCTACGCATTTATATAGAGGAGAC |
| | | AAGTCGTAACATGGTAAGT |

The data shown in FIGS. 1 and 2 could be presented as Tₘ^{WT}-Tₘ^{MUT} scatter plots (FIG. 3), which were used as standard diagrams for determination of sample genotype. For an unknown sample, once the data of Tₘ^{WT} and Tₘ^{MUT} were obtained, the genotype could be assigned based on the region where the data point lied in the standard genotyping diagram.

For human genomic DNA (gDNA) samples, PCR amplification could be conducted to produce sufficient amount of the specific target sequence of the mtDNA gene. The primer pairs were designed to flank the two mutation sites. Tables 3 and 4 show the PCR primers and thermal cycling parameters. Following PCR, two aliquots of the PCR products could be directly mixed with WT and MUT nanoprobes, respectively, and Tₘ values of the hybrids of WT probe/amplicon and MUT probe/amplicon could be measured. The obtained data point (Tₘ^{WT}, Tₘ^{MUT}) could be plotted in the standard genotyping diagram to determine the sample's genotype.

TABLE 3
PCR primers for target sequence amplification
(amplicon size: 250 nt).

| SEQ ID NO: | PCR primers | Sequence (5′ to 3′) |
|---|---|---|
| 2 | Forward | GAGTGCTTAGTTGAACAGGGC |
| 3 | Reverse | GGGTTTGGGGCTAGGTTTAG |

TABLE 4
Thermal cycler protocol for PCR amplification.

| | | |
|---|---|---|
| Initial denaturing | 95° C. | 3 min |
| 10-cycle touchdown | 95° C. | 20 sec |
| | 60° C., | 20 sec |
| | −0.5° C./cycle | |
| | 72° C. | 25 sec |
| 40-cycle amplification | 95° C. | 20 sec |
| | 55° C. | 20 sec |
| | 72° C. | 25 sec |
| End | 4° C. | Hold |

In summary, the inventors developed a dual-nanoparticle assay kit to gauge two mtDNA mutations related to aminoglycoside-induced hearing loss. The only equipment used was a standard thermal cycler, which allowed for cost-effective detection. The highly specific plasmonic nanoprobes ensured accurate genotyping based on colorimetric signals.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

1. Method of determining the presence of a mutation in human Mitochondrially Encoded 12S RNA (MTRNR1) gene (SEQ ID NO:1) in a sample, wherein the mutation is selected from the group consisting of 1555A>G and 1494C>T, and wherein the method comprises the steps of:
(a) amplifying at least part of the MTRNR1 gene, said part comprising the locus the mutation status of which is to be analyzed, in a polymerase chain reaction (PCR), using a pair of primers under conditions allowing such amplification to generate PCR amplification products (amplicons);
(b) contacting the amplicons in different test tubes with a plasmonic nanoprobe specific for the wild-type or mutant amplicons, respectively, said plasmonic nanoprobe comprising a plasmonic nanoparticle and a non-ionic oligonucleotide analog probe covalently coupled thereto, the oligonucleotide analog probe comprising a base sequence that is complementary to the wild-type or mutant amplicons, under conditions that allow the oligonucleotide analog probe and the amplicons to hybridize to each other, wherein the probe generates a detectable signal if hybridized to the amplicons that is distinguishable from the signal of the unhybridized probe;
(c) determining the presence of the mutation based on the determination of the melting temperature Tₘ of the hybrid of the nanoprobe and the amplicons.

2. The method according to claim 1, wherein the PCR used in step a) of the method is asymmetric PCR (aPCR).

3. The method according to claim 1, wherein the plasmonic nanoparticle comprised in the plasmonic nanoprobe as used in step b) of the method is a plasmonic gold nanoparticle.

4. The method according to claim 1, wherein the non-ionic oligonucleotide analog probe comprised in the plasmonic nanoprobe as used in step b) of the method is a morpholino oligonucleotide (MOR) probe.

5. The method according to claim 1, wherein the detectable signal generated in step b) of the method is the color of the assay solution that is indicative of whether the probe is hybridized to the amplicons or not.

6. The method according to claim 1, wherein the melting temperature determined in step c) is indicated by a color change caused by nanoprobe dissociation and subsequent aggregation.

7. The method according to claim 1, wherein the method further comprises isolating genomic DNA from the sample prior to step (a) of the method.

8. The method according to claim 1, wherein the method comprises using a nucleic acid molecule comprising at least part of the MTRNR1 gene comprising the 1555A>G and 1494C>T mutations as a positive control, and/or using a nucleic acid molecule comprising at least part of the MTRNR1 gene without said mutations as a negative control.

9. The method according to claim 1, wherein the PCR primers for use in the method have the nucleic acid sequences 5′-GAGTGCTTAGTTGAACAGGGC-3′ (SEQ ID NO:2) and 5′-GGGTTTGGGGCTAGGTTTAG-3′ (SEQ ID NO:3), and the oligonucleotide analog probes used are morpholino oligonucleotides having the nucleic acid sequence 5′-CGACTTGTCTCCTCTTTTTTTTTTT-3′ (SEQ ID NO:4) (specific for 1555A>G WT), 5′-CGACTTGCCTCCTCTTTTTTTTTTT-3′ (SEQ ID NO:5) (specific for 1555A>G MUT), 5′-TTGAGGAGGGTGACGTTTTTTTTTT-3′ (SEQ ID NO:6) (specific for 1494C>T WT) or 5′-TTGAGGAGAGTGACGTTTTTTTTTT-3′ (SEQ ID NO:7) (specific for 1494C>T MUT).

10. The method according to claim 1, wherein the morpholino oligonucleotides are modified with disulfide amide at the 3′ terminal.

11. The method according to claim 1, wherein the method is for use in determining the predisposition of a subject to a disease or disorder associated with the mutations of the MTRNR1 gene such as determining the risk of the subject to Aminoglycoside-induced hearing loss.

12. Kit for determining the presence of a mutation in human Mitochondrially Encoded 12S RNA (MTRNR1) gene (SEQ ID NO:1) in a sample, wherein the mutation is selected from the group consisting of 1555A>G and 1494C>T, and wherein the kit comprises a pair of PCR primers and a pair of plasmonic nanoprobes for use in a method of determining the presence of the mutation in the MTRNR gene in a sample, wherein the method comprises the steps of:
(a) amplifying at least part of the MTRNR1 gene, said part comprising the locus the mutation status of which is to be analyzed, in a polymerase chain reaction (PCR), using a pair of primers under conditions allowing such amplification to generate PCR amplification products (amplicons);
(b) contacting the amplicons in different test tubes with a plasmonic nanoprobe specific for the wild-type or mutant amplicons, respectively, said plasmonic nanoprobe comprising a plasmonic nanoparticle and a non-ionic oligonucleotide analog probe covalently coupled thereto, the oligonucleotide analog probe comprising a base sequence that is complementary to the wild-type or mutant amplicons, under conditions that allow the oligonucleotide analog probe and the amplicons to hybridize to each other, wherein the probe generates a detectable signal if hybridized to the amplicons that is distinguishable from the signal of the unhybridized probe;
(c) determining the presence of the mutation based on the determination of the melting temperature Tₘ of the hybrid of the nanoprobe and the amplicons.

13. The kit according to claim 12, wherein the kit is designed to determine both of the 1555A>G and 1494C>T mutations and thus comprises four plasmonic nanoprobes.

14. The kit according to claim 12, wherein the kit comprises a pair of PCR primers having the nucleic acid sequences 5′-GAGTGCTTAGTTGAACAGGGC-3′ (SEQ ID NO:2) and 5′-GGGTTTGGGGCTAGGTTTAG-3′ (SEQ ID NO:3), and four plasmonic gold nanoparticle each respectively functionalized with a morpholino oligonucleotide having the nucleic acid sequence 5′-CGACTTGTCTCCTCTTTTTTTTTTT-3′ (SEQ ID NO:4) (specific for 1555A>G WT), 5′-CGACTTGCCTCCTCTTTTTTTTTTT-3′ (SEQ ID NO:5) (specific for 1555A>G MUT), 5′-TTGAGGAGGGTGACGTTTTTTTTTT-3′ (SEQ ID NO:6) (specific for 1494C>T WT) or 5′-TTGAGGAGAGTGACGTTTTTTTTTT-3′ (SEQ ID NO:7) (specific for 1494C>T MUT), said morpholino oligonucleotides being modified with disulfide amide at the 3′ terminal.

15. The kit according to claim 12, wherein the kit is for use in determining the predisposition of a subject to a disease or disorder associated with the mutations of the MTRNR1 gene such as determining the risk of the subject to Aminoglycoside-induced hearing loss.
